SEQUENCE LISTING (13 sequences)

SEQ ID NO: 1 - protein, 450 aa, Acanthamoeba polyphaga mimivirus
NDDNLLVLGIGISVHKTDGVLRFEKYCQAHNLQYMIVGEGKKWNGGNLESEAGGGQKINELLIALESIKDNKLIVVCDTYDLIPLSGPEEILRKYRFLTPDNKVVFSSELYCWPDASLVERYPKVDTKYKYLNSGAFMGYRDDIYEMIKNGVKDRDDDQLFFSIKFIETDKIVLDYKCELFQAMYRCNSDLVVHKNRIFNGYTNSYPVFAHGNGPAKKLLNHMEGYFMTEPIDGSSNTINTFKLDNEPKVFFALYVDSNDLSALKQFLGKVASIQYGNKVIYLYDRSDNEQNRKLIQISYPNYHTGVTKYVFDDFKKSDAQFYFLLEQNCIITKKDILHELIMQVKDNHRVISPMIGYEQNSTRTNFWGDIEDGYYKRSENYLDLAKHKVRGLWNVPYVYGVILMHESVVRNWDLSMVKYNDKDMDLCFSLRKHTIFMYMINNNNYGYMV

SEQ ID NO: 2 - protein, 484 aa, Homo sapiens
NPEKLLVITVATAETEGYLRFLRSAEFFNYTVRTLGLGEEWRGGDVARTVGGGQKVRWLKKEMEKYADREDMIIMFVDSYDVILAGSPTELLKKFVQSGSRLLFSAESFCWPEWGLAEQYPEVGTGKRFLNSGGFIGFATTIHQIVRQWKYKDDDDDQLFYTRLYLDPGLREKLSLNLDHKSRIFQNLNGALDEVVLKFDRNRVRINVAYDTLPIVVHGNGPTKLQLNYLGNYVPNGWTPEGGCGFCNQDRRTLPGGQPPPRVFLAVFVEQPTPFLPRFLQRLLLLDYPPDRVTLFLHNNEVFHEPHIADSWPQLQDHFSAVKLVGPEEALSPGEARDMAMDLCRQDPECEFYFSLDADAVLTNLQTLRILIEENRKVIAPMLSRHGKLWSNFWGALSPDEYYARSEDYVELVQRKRVGVWNVPYISQAYVIRGDTLRMELPQRDVFSGSDTDPDMAFCKSFRDKGIFLHLSNQHEFGRLLVRI

SEQ ID NO: 3 - protein, 228 aa, Acanthamoeba polyphaga mimivirus
NDDNLLVLGIGISVHKTDGVLRFEKYCQAHNLQYMIVGEGKKWNGGNLESEAGGGQKINELLIALESIKDNKLIVVCDTYDLIPLSGPEEILRKYRFLTPDNKVVFSSELYCWPDASLVERYPKVDTKYKYLNSGAFMGYRDDIYEMIKNGVKDRDDDQLFFSIKFIETDKIVLDYKCELFQAMYRCNSDLVVHKNRIFNGYTNSYPVFAHGNPAKKLLNHMEGYFMT

SEQ ID NO: 4 - protein, 203 aa, Acanthamoeba polyphaga mimivirus
PKVFFALYVDSNDLSALKQFLGKVASIQYGNKVIYLYDRSDNEQNRKLIQISYPNYHTGVTKYVFDDFKKSDAQFYFLLEQNCIITKKDILHELIMQVKDNHRVISPMIGYEQNSTRTNFWGDIEDGYYKRSENYLDLAKHKVRGLWNVPYVYGVILMHESVVRNWDLSMVKYNDKDMDLCFSLRKHTIFMYMINNNNYGYMV

SEQ ID NO: 5 - protein, 5 aa, Homo sapiens
TLPGG

SEQ ID NO: 6 - protein, 14 aa, Homo sapiens
EEALSPGEARDMAM

SEQ ID NO: 7 - protein, 481 aa, synthetic construct
TDKLLVITVATKESDGFHRFMQSAKYFNYTVKVLGMGEEWRGGDGINSIGGGQKVRLLKEVMEKYRDQDDLVVMFTECFDVIFAGGPEEILKKFQKFNHKVVFAADGILWPDKRLADKYPVVHIGKRYLNSGGFIGYAPYVYRIVQQWNLQDNDDDQLFYTKVYIDPHKREKINITLDHKCKIFQTLNGAVDEVVLKFENGKARAKNTLYNTLPVIIHGNGPTKILLNYFGNYVPNSWTQDNGCTLCEFDTVDLSAVDVHPNVLIGVFIEQPTPFLPEFLDSLLTLDYPKEALKLFIHNKEVYHEKDIKKFFDKAKHEIKTIKIVGPEENLSEAEARNMAMDFCRQDEKCDYYFSVDADVVLTNPRTLKILIEQNRKIIAPLVTRPGKLWSNFWGALSPDGYYARSEDYVDIVQGKRVGIWNVPYMANVYLIKGETLRSEMNERNYFVRDKLDPDMALCRNAREMGVFMYVSNRHEFGRLL

SEQ ID NO: 8 - protein, 502 aa, synthetic construct
TDKLLVITVATKESDGFHRFMQSAKYFNYTVKVLGMGEEWRGGDGINSIGGGQKVRLLKEAMEKYKDQEDLVIMFTECFDVIFAGGPEELLKKFQKFNHKVVFAADGILWPDKRLADKYPVVHEGKRYLNSGGFIGYAPYVYRIVQQWNLQDNDDDQLFYTKVYIDPDKREKLNITLDHKCKIFQTLNGAVDEVVLKFENGKARARNTVYDTLPVIIHGNGPTKILLNYFGNYVPNAWTQDNGCTLCEFDTVDLSAVDVYPNVLIAIFIEQPTPFLPEFLDRLLTLDYPKERLKLFIHNNVEYHEKDIKKFFDKAKHEIKTIKIVGPEENLSEAEARNMAMDFCRQDPDCDYYFSVDADVVLTNPQTLKILIEQNRKIIAPLVTRPGKLWSNFWGALSPDGYYARSEDYVDIVQGKRVGIWNVPYMSHVYLIKGETLRSEMNERNYFVRDKLDPDMAWCRNIREMTLQREKDSPTPETFQMLSPPKGIFMYVSNRHEFGRLL

SEQ ID NO: 9 - protein, 225 aa, synthetic construct
LEVLFQGPGSTSHYNNDLWQIFDNPDDWKEKYIHPDYSKIFKENIVEQPCPDVYWFPIFTEKFCDELVEEMEHYGKWSGGKHHDSRISGGYENVPTDDIHMRQVGLEKVWLHFIREYIAPVTEKVFPGYYTKGFALLNFVVKYSPERQRSLRPHHDASTYTINIALNQVGEDFEGGGCRFLRYNCSIENPRKGWVFMHPGRLTHLHEGLPVTNGTRYIAVSFIDP

SEQ ID NO: 10 - protein, 23 aa, synthetic construct
MGSSHHHHHHSSGLEVLFQGPGS

SEQ ID NO: 11 - protein, 17 aa, Homo sapiens
GISLKGEEGIMGFPGLR

SEQ ID NO: 12 - protein, 895 aa, Acanthamoeba polyphaga mimivirus
MISRTYVINLARRPDKKDRILAEFLKLKEKGVELNCVIFEAVDGNNPEHLSRFNFKIPNWTDLNSGKPMTNGEVGCALSHWSVWKDVVDCVENGTLDKDCRILVLEDDVVFLDNFMERYQTYTSEITYNCDLLYLHRKPLNPYTETKISTHIVKPNKSYWACAYVITYQCAKKFMNANYLENLIPSDEFIPIMHGCNVYGFEKLFSNCEKIDCYAVQPSLVKLTSNAFNDSETFHSGSYVPSNKFNFDTDKQFRIVYIGPTKGNSFHRFTEYCKLYLLPYKVIDEKETNDFVSLRSELQSLSEQDLNTTLMLVVSVNHNDFCNTIPCAPTNEFIDKYKQLTTDTNSIVSAVQNGTNKTMFIGWANKISEFINHYHQKLTESNAETDINLANLLLISSISSDFNCVVEDVEGNLFQLINEESDIVFSTTTSRVNNKLGKTPSVLYANSDSSVIVLNKVENYTGYGWNEYYGYHVYPVKFDVLPKIYLSIRIVKNANVTKIAETLDYPKELITVSISRSEHDSFYQADIQKFLLSGADYYFYISGDCIITRPTILKELLELNKDFVGPLMRKGTESWTNYWGDIDPSNGYYKRSFDYFDIIGRDRVGCWNVPYLASVYLIKKSVIEQVPNLFTENSHMWNGSNIDMRLCHNLRKNNVFMYLSNLRPYGHIDDSINLEVLSGVPTEVTLYDLPTRKEEWEKKYLHPEFLSHLQNFKDFDYTEICNDVYSFPLFTPAFCKEVIEVMDKANLWSKGGDSYFDPRIGGVESYPTQDTQLYEVGLDKQWHYVVFNYVAPFVRHLYNNYKTKDINLAFVVKYDMERQSELAPHHDSSTYTLNIALNEYGKEYTAGGCEFIRHKFIWQGQKVGYATIHAGKLLAYHRALPITSGKRYILVSFVN

SEQ ID NO: 13 - protein, 455 aa, Acanthamoeba polyphaga mimivirus
MEQSNNDDNLLVLGIGISVHKTDGVLRFEKYCQAHNLQYMIVGEGKKWNGGNLESEAGGGQKINELLIALESIKDNKLIVVCDTYDLIPLSGPEEILRKYRFLTPDNKVVFSSELYCWPDASLVERYPKVDTKYKYLNSGAFMGYRDDIYEMIKNGVKDRDDDQLFFSIKFIETDKIVLDYKCELFQAMYRCNSDLVVHKNRIFNGYTNSYPVFAHGNGPAKKLLNHMEGYFMTEPIDGSSNTINTFKLDNEPKVFFALYVDSNDLSALKQFLGKVASIQYGNKVIYLYDRSDNEQNRKLIQISYPNYHTGVTKYVFDDFKKSDAQFYFLLEQNCIITKKDILHELIMQVKDNHRVISPMIGYEQNSTRTNFWGDIEDGYYKRSENYLDLAKHKVRGLWNVPYVYGVILMHESVVRNWDLSMVKYNDKDMDLCFSLRKHTIFMYMINNNNYGYMV

(19) United States
(12) Patent Application Publication
(10) Pub. No.: US 2024/0336947 A1
(43) Pub. Date: Oct. 10, 2024

(54) METHOD TO PRODUCE COLLAGEN AS THERAPEUTICS AND BIOMATERIALS

(71) Applicant: University of Kentucky Research Foundation, Lexington, KY (US)

(72) Inventor: Houfu Guo, Lexington, KY (US)

(21) Appl. No.: 18/626,636

(22) Filed: Apr. 4, 2024

Related U.S. Application Data
(60) Provisional application No. 63/494,023, filed on Apr. 4, 2023.

(51) Int. Cl. C12P 21/02; C12N 9/10

(52) U.S. Cl. CPC C12P 21/02; C12N 9/1051; C12Y 204/01066

(57) ABSTRACT

Humans and Acanthamoeba Polyphaga Mimivirus share numerous homologous genes, including collagens and collagen-modifying enzymes. To explore the homology, a genome-wide comparison was performed between human and mimivirus using DELTA-BLAST (Domain Enhanced Lookup Time Accelerated BLAST) and identified 190 new mimiviral proteins that share homology with 1236 human proteins. To gain functional insights into mimiviral proteins, the human protein homologs were organized into Gene Ontology (GO) and REACTOME pathways to build a functional network. Collagen and collagen-modifying enzymes form the largest subnetwork with most nodes. Further analysis of this subnetwork identified a putative collagen glycosyltransferase R699. Protein expression test suggested that R699 is highly expressed in E coli, unlike the human collagen-modifying enzymes. Enzymatic activity assays showed that R699 catalyzes the conversion of galactosyl-hydroxylysine to glucosyl-galactosyl-hydroxylysine on collagen using UDP-glucose as a sugar donor, suggesting R699 is a mimiviral collagen galactosylhydroxylysyl glucosyltransferase (GGT). Structural study of R699 produced the first crystal structure of a collagen GGT with uridine diphosphate glucose (UDP-Glc). Sugar moiety of the UDP-Glc resides in a previously unrecognized pocket. Mn²⁺ coordination and nucleoside-diphosphate binding site are conserved among GGT family members and critical for R699's collagen GGT activity. To facilitate further analysis of human and mimiviral homologous proteins, we presented an interactive and searchable genome-wide comparison app for quickly browsing of human and Acanthamoeba Polyphaga Mimivirus homologs, which is available at RRID Resource ID: SCR_022140 or guolab.shinyapps.io/app-mimivirus-publication/.

### CROSS-REFERENCE TO RELATED APPLICATIONS

The present application claims priority to U.S. Provisional Patent Application 63/494,023, filed Apr. 4, 2023, the content of which is hereby incorporated by reference in its entirety.

### GOVERNMENT SUPPORT

This invention was made with government support under R00CA225633 awarded by The National Institutes of Health. The government has certain rights to the invention.

### FIELD

The present disclosure concerns methods for producing expressing and producing collagens.

### BACKGROUND

Collagens are the most abundant extracellular matrix proteins representing ˜30% of the total proteins by mass. At least 28 families of collagen are present in humans. They are produced by collagen modifying enzymes to generate a matrix to support cell adhesion and communication, playing critical roles in development, connective tissue disorders, cancer, and fibrosis. Because of their critical interactions with cells, availability and biocompatibility, animal-derived collagens have already widely used as coating materials for tissue culture and biomaterials for surgical and medical procedures in the life science and biomedical community. However, it is desirable to produce recombinant human collagen since animal-derived collagen is different from humans and may contain pathogens such as viruses. The bacterial protein expression system is preferred for this purpose because it is highly scalable, cost-effective, and highly adaptable to produce different collagens. Moreover, unlike animal cells that produce multiple types of collagens simultaneously, bacteria do not have endogenous collagen, so single type of collagen may be produce in bacteria with no endogenous collagen contamination. Unfortunately, a bacterial collagen expression system is currently not available because most of the human collagen modifying enzymes cannot be produced in bacteria.

### SUMMARY

The present disclosure concerns systems and/or methods for modifying collagen(s). In aspects, the present disclosure relates to the identification of collagen modifying enzymes in the mimiviral genome. In some aspects, the systems and/or methods concern contacting mimiviral enzymes and/or derivatives and/or mutants thereof with a collagen, such as a human collagen, whereby, the collagen is then post-translationally modified by the activity of the enzyme.

A 1^{st} aspect of the present disclosure, either alone or in combination with any other aspect set forth herein, concerns a system for producing collagen comprising a bacterial cell expressing an enzyme with at least 85% identity to an amino acid sequence as set forth in at least one of SEQ ID NOs: 1, 7, 8, 9, 12, or 13.

A 2^{nd} aspect of the present disclosure, either alone or in combination with any other aspect set forth herein, concerns the system of the 1^{st} aspect, wherein the bacterial cell expresses SEQ ID NO: 1.

A 3^{rd} aspect of the present disclosure, either alone or in combination with any other aspect set forth herein, concerns the system of the 1^{st} aspect, wherein the bacterial cell expresses SEQ ID NO: 13.

A 4^{th} aspect of the present disclosure, either alone or in combination with any other aspect set forth herein, concerns the system of the 1^{st} aspect, wherein the bacterial cell expresses SEQ ID NO: 7.

A 5^{th} aspect of the present disclosure, either alone or in combination with any other aspect set forth herein, concerns the system of the 1^{st} aspect, wherein the bacterial cell expresses SEQ ID NO: 8.

A 6^{th} aspect of the present disclosure, either alone or in combination with any other aspect set forth herein, concerns the system of the 1^{st} aspect, wherein the bacterial cell expresses SEQ ID NO: 9.

A 7^{th} aspect of the present disclosure, either alone or in combination with any other aspect set forth herein, concerns the system of the 1^{st} aspect, wherein the bacterial cell is an Escherichia coli cell.

An 8^{th} aspect of the present disclosure, either alone or in combination with any other aspect set forth herein, concerns the system of the 1^{st} aspect, wherein the bacterial cell further expresses a human collagen protein or fragment thereof.

A 9^{th} aspect of the present disclosure, either alone or in combination with any other aspect set forth herein, concerns the system of the 1^{st} aspect, further comprising a human collagen protein.

A 10^{th} aspect of the present disclosure, either alone or in combination with any other aspect set forth herein, concerns a method for modifying a collagen protein comprising contacting a collagen protein with a recombinant galactosylhydroxylysyl glucosyltransferase (GGT) derived from Acanthamoeba polyphaga mimivirus.

An 11^{th} aspect of the present disclosure, either alone or in combination with any other aspect set forth herein, concerns the method of the 10^{th} aspect, wherein the GGT is selected from the group consisting of SEQ ID NOs: 1, 7, 8, 9, 12, and 13.

A 12^{th} aspect of the present disclosure, either alone or in combination with any other aspect set forth herein, concerns the method of the 11^{th} aspect, wherein the GGT comprises SEQ ID NO: 13.

A 13^{th} aspect of the present disclosure, either alone or in combination with any other aspect set forth herein, concerns the method of the 10^{th} aspect, wherein the GGT is recombinantly expressed in a bacterial cell.

A 14^{th} aspect of the present disclosure, either alone or in combination with any other aspect set forth herein, concerns the method of the 13^{th} aspect, wherein the bacterial cell is an Escherichia coli cell.

An 15^{th} aspect of the present disclosure, either alone or in combination with any other aspect set forth herein, concerns the method of the 13^{th} aspect, wherein the GGT is expressed from a nuclei acid encoding the GGT within the bacterial cell.

### BRIEF DESCRIPTION OF THE DRAWINGS

FIGS. 1A-C shows comparative genomic analysis of humans and mimivirus. FIG. 1A shows functional enriched Gene Ontology (GO) and REACTOME pathways that are shared between humans and mimivirus. We performed a genome-wide search of homologous proteins in human genome using the translation products of mimiviral ORFs as queries. This search identified 4123 unique human RefSeq records that were organized into GO and REACTOME pathways. Gene ontology networks were built based on the results from pathway analysis and visualized using Cytoscape 3.8.2. Collagen related networks were highlighted with a dashed black square. FIG. 1B shows collagen related pathways form the largest subnetwork with the most nodes. Top 20 GO enriched biological processes were shown. Collagen-related pathways are the major components of Extracellular matrix organization and Extracellular structure organization. Collagen related pathways were highlighted in bold. FIG. 1C shows subnetwork analysis shows the organization of collagen-related pathways.

FIG. 2 shows schematics showing collagen lysyl PTMs pathway and its homology to mimiviruses. Sequence alignments suggested that mimiviral L230, R699 and R655 are homologous to human collagen lysyl modifying enzymes involved in collagen crosslinking and Hyl-O-linked glycosylation. A newly identified mimiviral putative collagen glycosyltransferase R699 shares higher amino acid sequence identity (˜30%) with human collagen GGTs than hydroxylysyl galactosyltransferases (GLT25D1 and GLT25D2). Homology was indicated with black lines (solid black lines if percentage of QuerySpan was higher than 70%, and dashed black lines if percentage of QuerySpan fell between 25% and 70%) and the percentage of amino acid sequence identity was shown.

FIGS. 3A-H shows R699 is a GGT. FIG. 3A shows SDS-polyacrylamide gel electrophoresis of R699 protein after immobilized metal affinity chromatography (IMAC) with nickel resin (N), HRV 3C protease cleavage (P), reverse IMAC (R). R699 was purified close to homogeneity after 3-step purification. The approximate size of the recombinant protein was indicated with an arrow. Gel image was recolored. FIG. 3B shows R699 GGT activity was assayed using an adenosine triphosphate-based luciferase assay. Substrate was galactosylhydroxylysine. GGT activity was measured by detecting UDP production. Mean±SD of 3 replicates, p values, two-tailed Student's t test. FIG. 3C and FIG. 3D show R699 GGT assay was performed using UDP-[UL-¹³C₆] glucose. Carbon-13 labeled glucosylgalactosyl-hydroxylysine ([¹³C]GlcGal-Hyl) was confirmed with LC-MS analysis. The amount of [¹³C]GlcGal-Hyl was determined based on LC peaks in FIG. 3C using MultiQuant software (SCIEX). Chromatograms 3C and bar graph 3D were plotted using custom R scripts. Galactosyl-hydroxylysine was shown as Gal-Hyl. n=1. FIG. 3E shows sequence alignment of R699 (SEQ ID NO: 1) with human LH3 (SEQ ID NO: 2). Residues within the GGT and accessory (AC) domains were labeled (SEQ ID NO: 3 and SEQ ID NO: 4). Asp190 and Asp191 in poly-Asp repeat and Trp145 were indicated with arrows (residue number based on LH3 sequence). The interdomain loop deletion (SEQ ID NO: 5) and the largest deletion in R699 were highlighted. FIG. 3F shows SDS-polyacrylamide gel electrophoresis of R699 wild type (WT) and mutant recombinant proteins. The approximate size of the recombinant proteins was indicated with an arrow. Gel image was recolored. FIG. 3G shows GGT activity of WT and mutant R699 recombinant proteins was assayed using Gal-Hyl as substrate. The readout of the assay is adenosine triphosphate production which was detected using an adenosine triphosphate-based luciferase assay. Mean±SD of 3 replicates, p values, two-tailed Student's t test. FIG. 3H shows circular dichroism spectrometry of wild-type R699 (WT) and R699 mutants.

FIGS. 4A-E shows R699 is a collagen GGT. FIG. 4A shows Type IV collagen that had been pre-treated with wild-type (+) protein-glucosylgalactosylhydroxylysine glucosidase (PGGHG) or sham-treated (−) was analyzed using SDS-polyacrylamide gel electrophoresis. Gel image was recolored. FIG. 4B shows R699 collagen GGT activity was assayed. Substrate was deglucosylated type IV collagen from A and GGT activity was measured similarly as in FIG. 3B. Mean±SD of 3 replicates, p values, two-tailed Student's t test. FIG. 4C shows MS/MS analysis of type IV collagen. R699 GGT assay was performed using deglucosylated type IV collagen and UDP-[UL-¹³C₆] glucose. Peptides containing galactosyl-hydroxylysine (Gal-Hyl, top) or Carbon-13 labeled glucosyl-galactosylhydroxylysine ([¹³C]GlcGal-Hyl, bottom) (SEQ ID NO: 11) were detected by MS/MS. The spectra exhibit peaks corresponding to b and y ion series from fragmentation of each peptide. Peptide sequences with the identified fragment ions were indicated in the upper right. The b and y ions were labeled in the spectra and indicated on the peptide sequence in upper right. FIG. 4D shows zoom-in of spectra in 4C showing y15++. Peptide sequences, b and y ions were labeled similarly as in 4C. The locations of y15++ in the spectra and peptide sequences were highlighted with arrows. FIG. 4E shows the details about peptide sequences, modifications, and abundance were summarized.

FIGS. 5A-D shows human and mimivirus homology tool features and functionalities. FIG. 5A shows an interactive tool was established for easily searching and browsing of human and mimiviral homolog proteins. FIG. 5B shows bar graph showing the overall distribution of homologous proteins. FIG. 5C shows after performing search, clicking Excel (highlighted with a blue square) to download a list of human or mimivirus homologous proteins. FIG. 5D shows clicking on the query_id to show the details of the query and search (the Gene ID, symbol, description and sequence). Under the section of Data Table of All Hits, an excel file with the details of the search is available for download.

### DESCRIPTION

The present disclosure concerns a bacterial expression system for collagen production. In some aspects, the present disclosure is based on bioengineering of human collagen modifying enzymes and expressing them in bacteria to generate a collagen-producing bacterium. In some aspects, the present disclosure concerns the engineering of 2 human collagen modifying enzymes and the expression and/or production thereof a bacteria cell with robust enzymatic activities. In some aspects, the bacterial expressed enzymes can then be used as part of a bacterial system for producing recombinant collagen, such as recombinant human collagen. Human recombinant collagen generated with this system and the methods set forth herein may be used for further applications, such as tissue culture and use as biological implants. Since certain Rheumatoid Arthritis (RA) is caused by autoimmune responses to glycosylated type II collagen, the systems and methods herein may also be used to produce glycosylated type II collagen and collagen fragments as diagnostic and therapeutic reagents for Rheumatoid Arthritis. The present system and methods provide the ability to generate tailor-designed collagen and collagen fragments to meet the unique need of individual patients.

Type IV collagen contains the N-terminal 7S, a central triple-helical domain, and the globular C-terminal NC1. To perform their functions, collagens acquire a series of specific post-translational modifications (PTMs) during biosynthesis⁴. Collagen prolyl 4-hydroxylation catalyzed by collagen prolyl 4-hydroxylases is critical for stabilizing the triple-helical structure of collagens⁵. Prolyl 3-hydroxylases catalyze prolyl 3-hydroxylation and defects in this minor modification are associated with recessive osteogenesis imperfecta. A series of lysine (Lys) PTMs of collagens are critical for the stability of collagen fibrils. In the cells, Lys residues in the sequences of X-Lys-Gly (helical domain) and X-Lys-Ala/Ser (telopeptides) can be hydroxylated by lysyl hydroxylases 1-3 (LH1-3) to form 5-hydroxylysine (Hyl). It is generally accepted that LH1 is the main LH for the helical domain and LH2 for the telopeptides. Certain Hyl residues in the collagen helical domain are galactosylated by glycosyltransferase 25 domain containing 1 and 2 (GLT25D1 and GLT25D2) and then glucosylated by lysyl hydroxylases to form a unique Hyl-O-linked glycosylation with a mono- or di-saccharide. LH3 was believed to be the only galactosylhydroxylysyl glucosyltransferase (GGT) catalyzing collagen glucosylation, however, recent studies suggested LH1 and LH2 have GGT activities as well. Collagen prolyl and lysyl PTMs are tightly regulated during the development and their alterations lead to various diseases. For instance, mutations in the gene encoding LH2 result in Bruck syndrome II a rare osteogenesis imperfecta with joint contracture, but hyper LH2 activities contribute to fibrosis and cancer growth and metastasis.

Besides multicellular animals, collagen-like proteins and collagen-modifying enzymes are also highly conserved across species and have been found in certain fungi, bacteria, and viruses such as mimivirus. Since the initial release of the mimiviral genome (Acanthamoeba Polyphaga Mimivirus), studies have identified 7 mimiviral collagen genes and 2 mimiviral collagen-modifying enzyme genes that encode three enzymes, including collagen prolyl hydroxylase, collagen lysyl hydroxylase, and collagen hydroxylysyl glucosyltransferase. Structural and functional studies of mimiviral collagen lysyl hydroxylase provide insights into functions of the human collagen-modifying enzymes. Since collagen is widely used for tissue and biomaterial engineering, efforts have been made to generate recombinant collagens using different expression systems. Interestingly, a hydroxylated human collagen III fragment has been produced in Escherichia coli by coexpressing it with mimiviral collagen prolyl and lysyl hydroxylases. However, glycosylated human collagen is still unable to be produced in the bacterial expression system, at least due in part to the difficulty of expressing active human collagen glycosyltransferases in bacteria.

Mimivirus is the first giant virus discovered and is the prototype and best-characterized virus in the family. The initial mimiviral genome sequencing effort identified 917 protein-encoding genes. These genes play diverse functions in nucleotide and protein biosynthesis, including DNA replication, repair, transcription and translation. This effort also identified enzymes involved in various PTMs including 11 glycosyltransferases. As the sequencing technique advances, a later sequencing analysis identified 75 new genes and increased the mimiviral genes to exceed 1000. More recent work identified citric acid cycle and β-oxidation pathway genes in the Mimiviridae family. Since the release of the mimiviral genome sequence and the first search for its homology to other species, more than 50 mimiviral proteins have been expressed and characterized, which provides valuable insights into virology and raises questions regarding the definition of viruses. To facilitate the further study of mimiviral homologous proteins, a systematic search of mimiviral homologous proteins in humans was performed. Human and mimiviral proteins were compared at the genome-wide level using the DELTA-BLAST (Domain Enhanced Lookup Time Accelerated BLAST). Besides the initially identified 194 mimiviral ORFs that shared homology with human genes mainly involving in DNA and protein metabolism, 52 new mimiviral ORFs were found that may encode proteins with similarity to these of humans. Eight mimiviral collagen-like proteins (L71, L668, L669, R196, R238, R239, R240, and R241) and 4 putative mimiviral collagen-modifying enzymes (L230, L593, R655, and R699) were identified. To validate the results, a putative mimiviral collagen glycosyltransferase R699 was expressed and showed that R699 glucosylates both free galactosylhydroxylysine and collagen peptidyl galactosylhydroxylysine. These findings suggested that galactosylhydroxylysyl glucosyl-transferase is not restricted to the domains of life. Mimiviruses may have the ability to generate Hyl-O-linked glycosylation in a similar way as animals. Since mimiviral collagen modifying enzymes are stable in the bacterial expression systems, these enzymes, such as L230 and R699, may be useful to produce recombinant collagen to meet the biomedical research and clinical needs. Moreover, herein is established an interactive and searchable genome-wide comparison tool (RRID Resource ID: SCR_022140 or guolab.shinyapps.io/app-mimivirus-publication/). This user-friendly website helps users quickly browse the protein sequence homology between humans and mimivirus at the genome-wide level for querying new homologs and generating new hypotheses.

LH family members have 3-domain architectures: a domain for lysyl hydroxylase (LH) activity, a domain for galactosylhydroxylysyl glucosyltransferase (GGT) activity, and an accessory domain (AC). Mutated and/or truncations of LH proteins were performed. LH2 (lysyl hydroxylase isoform 2) was truncated and mutated to the following amino acid sequence:

(SEQ ID NO: 7)
TDKLLVITVATKESDGFHRFMQSAKYFNYTVKVLGMGEEWRGGDGINSIG
GGQKVRLLKEVMEKYRDQDDLVVMFTECFDVIFAGGPEEILKKFQKFNHK
VVFAADGILWPDKRLADKYPVVHIGKRYLNSGGFIGYAPYVYRIVQQWNL
QDNDDDQLFYTKVYIDPHKREKINITLDHKCKIFQTLNGAVDEVVLKFEN
GKARAKNTLYNTLPVIIHGNGPTKILLNYFGNYVPNSWTQDNGCTLCEFD
TVDLSAVDVHPNVLIGVFIEQPTPFLPEFLDSLLTLDYPKEALKLFIHNK
EVYHEKDIKKFFDKAKHEIKTIKIVGPEENLSEAEARNMAMDFCRQDEKC
DYYFSVDADVVLTNPRTLKILIEQNRKIIAPLVTRPGKLWSNFWGALSPD
GYYARSEDYVDIVQGKRVGIWNVPYMANVYLIKGETLRSEMNERNYFVRD
KLDPDMALCRNAREMGVFMYVSNRHEFGRLL
(referred to as LH2a-GGT-AC)

LH2b (which contains a fragment of exon 13a was truncated and mutated to provide the following amino acid sequence:

(SEQ ID NO: 8)
TDKLLVITVATKESDGFHRFMQSAKYFNYTVKVLGMGEEWRGGDGINSIG
GGQKVRLLKEAMEKYKDQEDLVIMFTECFDVIFAGGPEELLKKFQKFNHK
VVFAADGILWPDKRLADKYPVVHEGKRYLNSGGFIGYAPYVYRIVQQWNL
QDNDDDQLFYTKVYIDPDKREKLNITLDHKCKIFQTLNGAVDEVVLKFEN
GKARARNTVYDTLPVIIHGNGPTKILLNYFGNYVPNAWTQDNGCTLCEFD
TVDLSAVDVYPNVLIAIFIEQPTPFLPEFLDRLLTLDYPKERLKLFIHNN
VEYHEKDIKKFFDKAKHEIKTIKIVGPEENLSEAEARNMAMDFCRQDPDC
DYYFSVDADVVLTNPQTLKILIEQNRKIIAPLVTRPGKLWSNFWGALSPD
GYYARSEDYVDIVQGKRVGIWNVPYMSHVYLIKGETLRSEMNERNYFVRD
KLDPDMAWCRNIREMTLQREKDSPTPETFQMLSPPKGIFMYVSNRHEFGR
LL
(referred to as LH2b-GGT-AC)

A truncated mutant of the LH domain only of LH2 was also prepared with the following amino acid sequence:

(SEQ ID NO: 9)
LEVLFQGPGSTSHYNNDLWQIFDNPDDWKEKYIHPDYSKIFKENIVEQPC
PDVYWFPIFTEKFCDELVEEMEHYGKWSGGKHHDSRISGGYENVPTDDIH
MRQVGLEKVWLHFIREYIAPVTEKVFPGYYTKGFALLNFVVKYSPERQRS
LRPHHDASTYTINIALNQVGEDFEGGGCRFLRYNCSIENPRKGWVFMHPG
RLTHLHEGLPVTNGTRYIAVSFIDP
(referred to as LH2-LH)

In some aspects, the mutants may also include a hexa-histidine tag (His6) and/or an HRV 3C cleavage site. In some aspects, the following peptide may be appended to the amino and/or carboxyl terminus: MGSSHHHHHHSSGLEVLFQGPGS (SEQ ID NO: 10).

In some aspects, the present disclosure concerns methods and/or systems for modifying collagens, such as human collagens. In some aspects, the systems and/or methods include contacting a collagen, such as an expressed or a recombinant collagen, with a mimivirus enzyme or derivative as set forth herein, including GGT and/or LH fragments thereof, including LH2 and/or LH3 fragments. In some aspects, the methods include introducing and/or contacting a collagen with an enzyme as set forth in at least one of SEQ ID NOs: 1, 7, 8, 9, 12, and/or 13. In some aspects, the enzyme has at least 85% identity to the sequences as set forth in SEQ ID NOs: 1, 7, 8, 9, 12, and/13, including 86, 87, 88, 89, 90, 91, 92, 93, 94, 95, 96, 97, 98, 99, 99.1, 99.2, 99.3, 99.4, 99.5, 99.6, 99.7, 99.8, 99.9, and 100% identity. In some aspects, such may be expressed and/or purified from a bacterial cell, such as an Escherichia coli cell. In some aspects, the enzymes or derivatives thereof may be expressed in a bacterial cell through introducing a nucleic acid encoding the same into the bacterial cell. In some aspects, the nucleic acid(s) may be further modified to introduced appended tags or markers, such as fluorescent tags, hexahistidine tags, antibody recognition domains and the like. Nucleic acids and chimeric proteins, including vectors and plasmids for the expression in bacteria are understood in the art.

In some aspects, the collagen is one or more of type I, II, III, IV, V, VI, VII. VIII, IX, X, XI, XII, XIII, XIV, XV, XVI, XVII, XVIII, and/or XIX. In some aspects, the collagen can be expressed in the same cell as the enzyme or separately. The collagen can be a procollagen or a collagen gene under control of a vector with a promoter, such as a constitutively active or inducible promoter to drive expression thereof. Similarly, the enzymes can be expressed through a promoter, either constitutively active or inducible to drive expression thereof. Altered DNA sequences which may be used in accordance with the invention include deletions, additions or substitutions of different nucleotide residues resulting in a sequence that encodes the same or a functionally equivalent gene product. The gene product itself may contain deletions, additions or substitutions of amino acid residues within a collagen sequence, which result in a functionally equivalent collagen and/or enzyme

979 mimiviral ORFs were utilized to query human homologs in human non-redundant protein sequences using DELTA-BLAST. When using e-value<=0.01, hit span>=35 aa, % identical sequences>=0.25 as cutoffs, 322 queries resulted in at least 1 hit with 41520 hits in total. The search found 4123 unique human RefSeq records in total. Further analysis showed that the 4123 unique human RefSeq records are from 1236 unique human proteins. To identify mimiviral protein homologs using human protein queries, 4123 unique human RefSeq records were used that had been identified in the first round of analysis to search for mimiviral homologs. Using the same filtering standard, it was found that 3325 RefSeq queries or 1049 human proteins result in at least 1 hit and 58011 hits in total. This search found 307 mimiviral protein sequences, 265 of which overlap with the 322 mimiviral queries that had been started with. The most common motif shared by humans and mimivirus is ankyrin repeats. Besides the 48 mimiviral ankyrin repeats previously identified, these analyses added 33 more mimiviral proteins with ankyrin repeats.

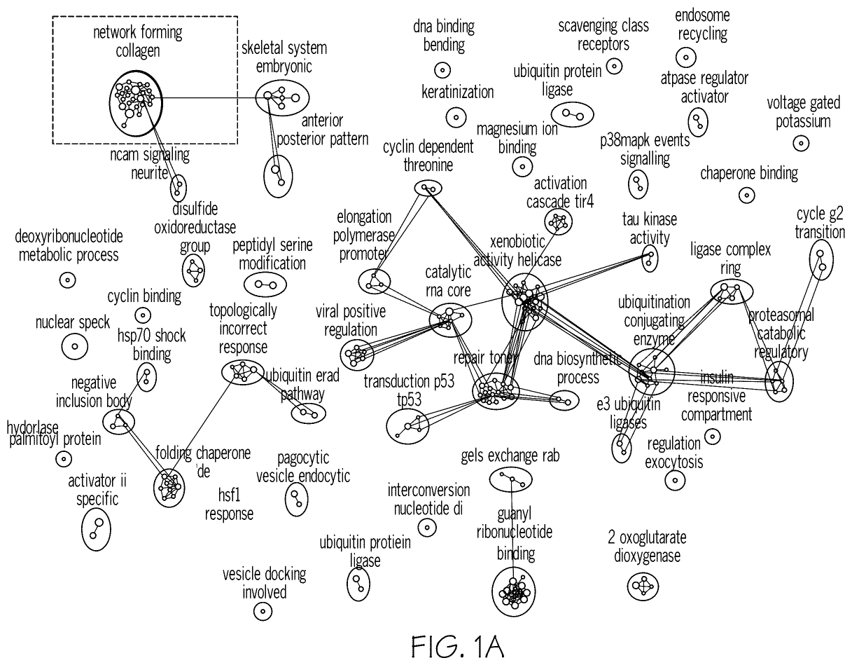

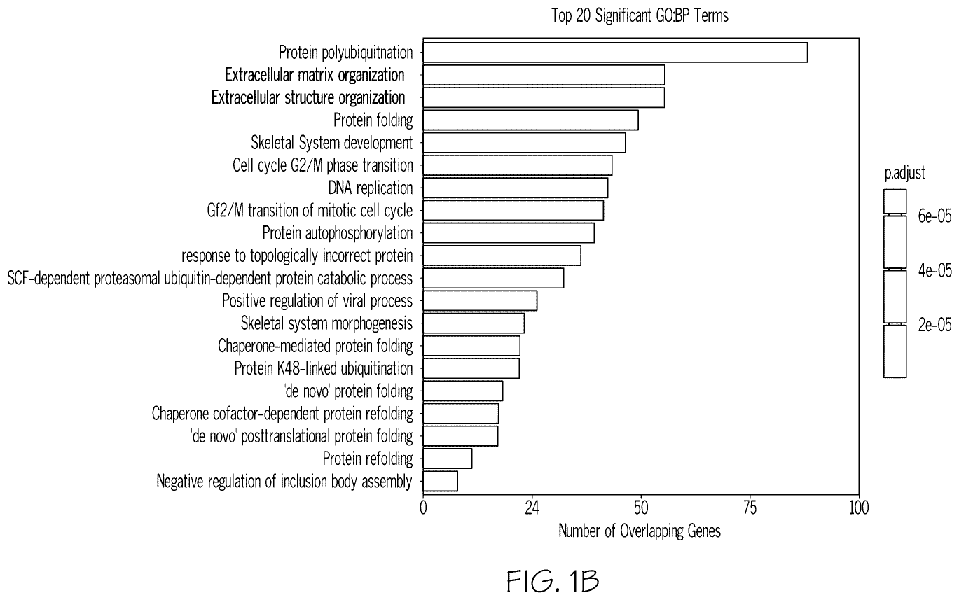

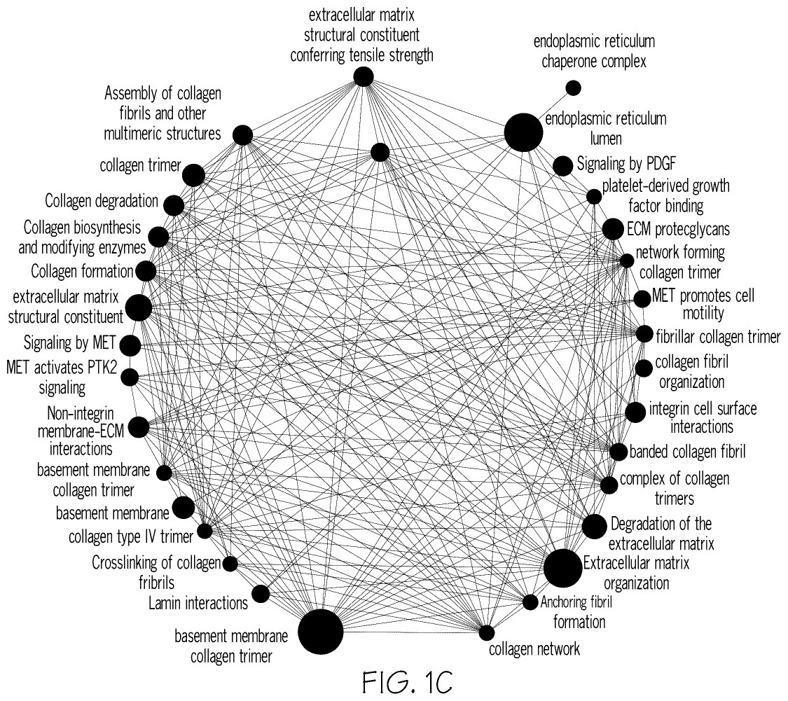

To identify the enriched pathways conserved between humans and mimivirus, GO and REACTOME pathway analysis was performed using an adjusted p-value<=0.05 as a cutoff. GO pathways were organized based on cellular component, molecular function, and biological process. Functional enriched GO and REACTOME pathways were then used to build gene ontology networks and visualized using Cytoscape 3.8.2 (FIG. 1A). This analysis identified 52 clusters involved in endocytosis, ubiquitination, DNA and collagen metabolism. The largest cluster with the most nodes is the network forming collagen composed of collagen and collagen-modifying enzymes (FIG. 1A). Collagen-related pathways rank high in both GO and REACTOME pathway analyses (FIG. 1B).

Homology in collagen and collagen-modifying enzymes

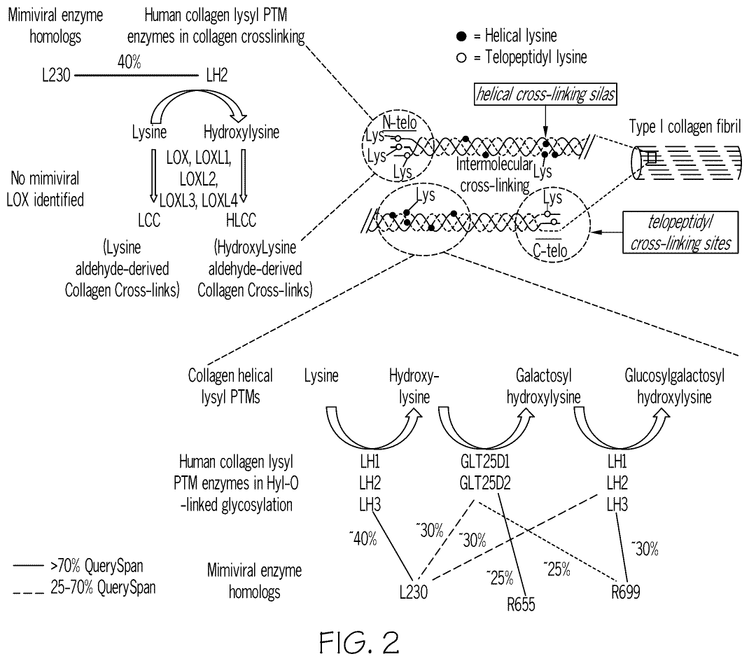

Subnetwork analysis on network forming collagen was performed since it is the largest network with most nodes (FIG. 1C). Subnetwork analysis showed that the human protein hits identified using mimiviral queries are involved in collagen biosynthesis and assembly. Our search identified 8 mimiviral collagens (L71, L668, L669, R196, R238, R239, R240, and R241) and 4 mimiviral collagen-modifying enzymes (L230 (SEQ ID NO: 12), L593, R655, and R699 (SEQ ID NO: 13)) (FIG. 2). Three of these proteins (R238, R655, and R699) have not been studied. Analyses suggest R238 is a collagen-like protein while R655 and R699 are putative collagen glycosyltransferases. Eight of the mimiviral collagen-related proteins identified by previous effort were not correctly annotated. For instance, 4 mimiviral collagen-like proteins (R196, L669, R239, and R241) had been annotated as PPE-repeat proteins and 2 putative collagen-modifying enzymes (L230 and R655) as LPS biosynthesis enzymes. Of these mis-annotated mimiviral proteins, L230 has been expressed and characterized as collagen telopeptidyl lysyl hydroxylase and collagen hydroxyl lysyl glucosyltransferase. Structural and mutagenesis analyses suggest that L230 lysyl hydroxylase domain forms a Fe²⁺-stabilized tail-to-tail homodimer, similar to human lysyl hydroxylase 2. For the mimiviral proteins that were not annotated during the initial release of the mimiviral genome, L71 has been confirmed to be a type of mimiviral collagen that may play a role in the pathogenesis of arthritis in humans. L593 was shown to hydroxylate human type III collagen proline residue and was used to generate a human recombinant collagen II fragment in a bacterial expression system. These results validate our search of the viral collagen and collagen-modifying enzymes and suggest that our analyses are robust and relevant. No lysyl oxidase or transglutaminase was identified, suggesting that mimiviral collagens are either crosslinked by host enzymes or not crosslinked at all.

R699 has collagen galactosylhydroxylysyl glucosyltransferase activity

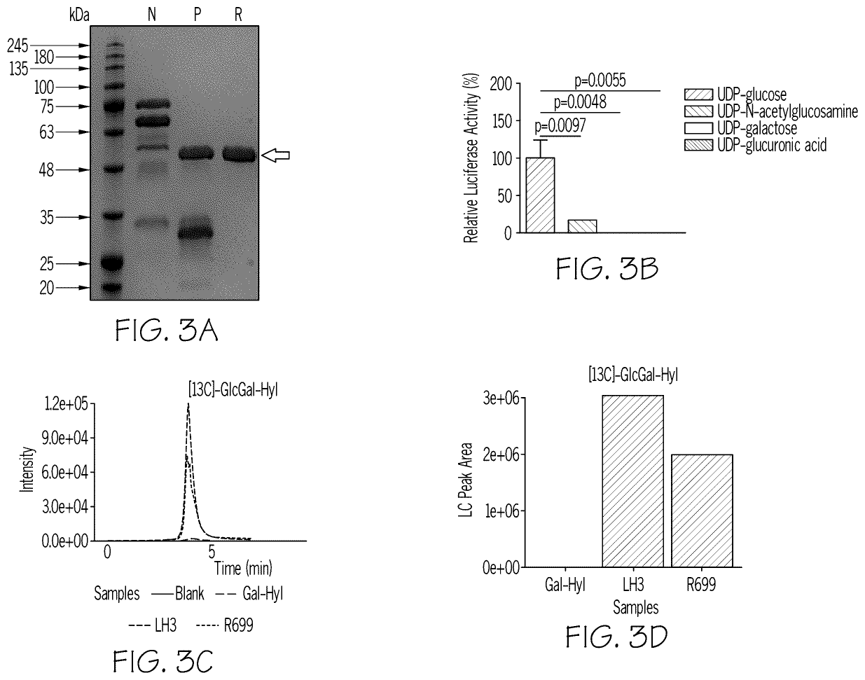

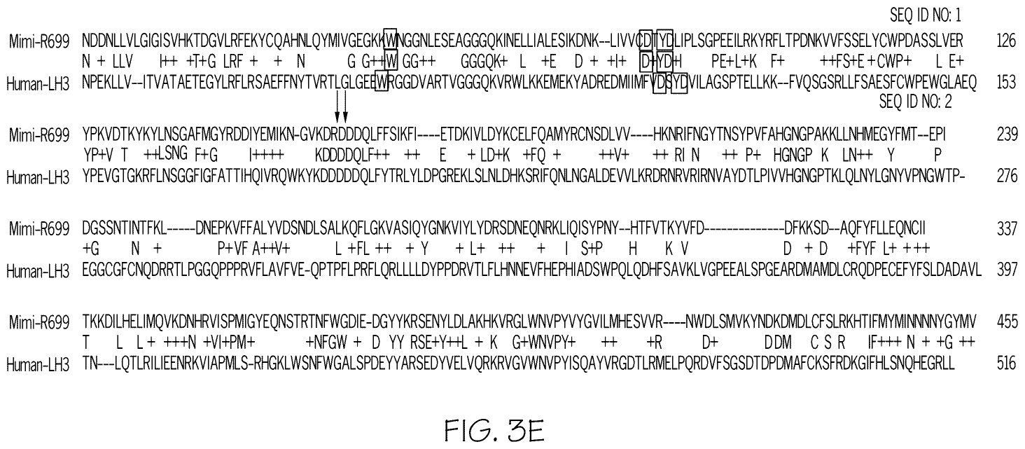

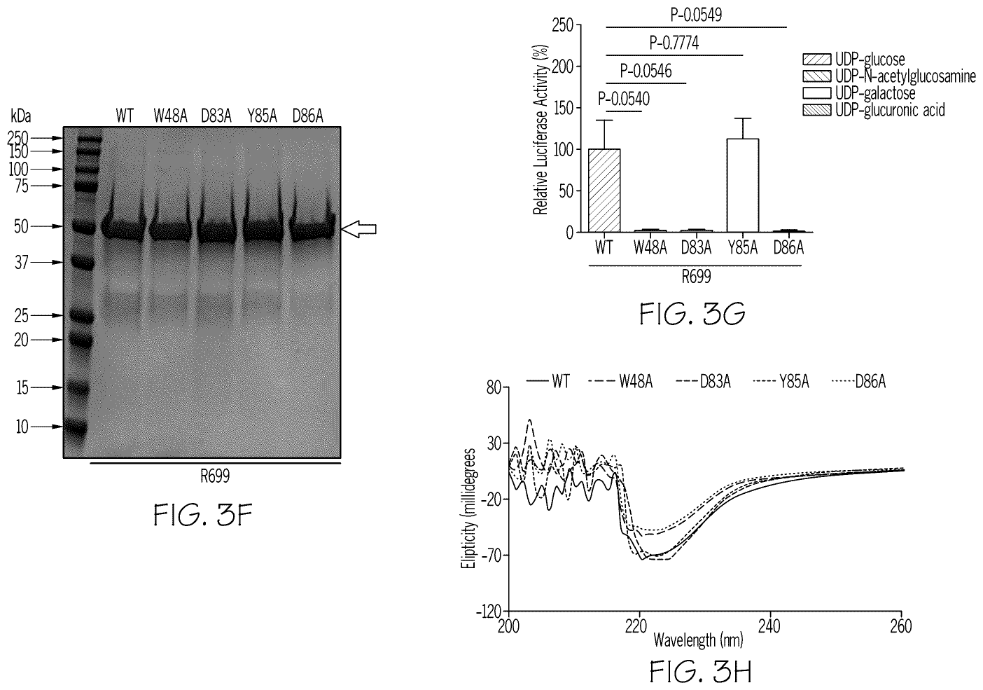

Of the two new putative collagen-modifying enzymes, R655 was speculated to be a putative mimiviral glycosyltransferase while little is known about R699. As a result, R699 was selected for further biochemical analyses. It was hypothesized that R699 is a collagen GGT because R699 shows a higher sequence similarity to human collagen GGTs than collagen hydroxylysyl galactosyltransferases (FIG. 2, 29.3% amino acid sequence identity with human LH2, E value=6e-60). To generate R699 recombinant protein for biochemical analyses, the R699 gene was synthesized with an HRV 3C protease cleavable N-terminal His₆ and mCherry tags and expressed it in Escherichia coli. It was found that R699 produces a stable soluble protein (FIG. 3A) after overnight Isopropyl β-D-1-thiogalactopyranoside-indued expression at 16° C. The Escherichia coli cells were lysed via sonication and purified the R699 protein with immobilized metal affinity chromatography (IMAC) using nickel resin. N-terminal His₆ and mCherry tags were cleaved by HRV 3C protease and removed by reversed IMAC. Highly purified R699 protein was obtained after the 3-step purification procedure (FIG. 3A) with a yield of ˜10 mg per liter of E. coli culture.

Given the moderate sequence similarity between R699 and human GGTs, it was hypothesized that R699 functions as a collagen GGT. To test this possibility, R699 was reacted with amino acid substrate galactosylhydroxylysine using UDP-glucose and 3 other sugar donors. Glycosylation was measured by detecting UDP production with a luciferase-based assay. Under these conditions, R699 showed robust activity with UDP-glucose but no other sugar donors (FIG. 3B). The GGT enzymatic activity assay was also performed using UDP-[UL-¹³C₆]-glucose as a sugar donor to confirm the glucosylation events by liquid chromatography-mass spectrometry (LC-MS) analysis. The glucosylation of galactosylhydroxylysine to [¹³C]-glucosylgalactosylhydroxylysine was detected in the presence of R699 (FIG. 3C, 3D) or a known collagen GGT (recombinant human LH3). [¹³C₆]-glucosylgalactosylhydroxylysine was not detected in buffer or galactosyl-hydroxylysine (Gal-Hyl) negative control.

By comparing with the LH3 catalytic domain, it was found that the R699 Mn²⁺-binding DXXD motif and UDP-binding Trp and Tyr residues are strictly conserved (FIG. 3E). Site-directed mutagenesis and enzymatic activity assay showed that DXXD is critical for R699's GGT activity (FIG. 3F,3G). Interestingly, Trp but not Tyr is critical for GGT activity (FIG. 3F,3G), suggesting Trp is the primary residue engaging UDP. Circular dichroism spectra suggested D83A and Y85A show similar spectra as the wild type (FIG. 3H) while W48A and D86A are slightly different, suggesting D83A and Y85A are not deleterious to R699 secondary structure.

Other residues critical for collagen GGT function are conserved in R699 as well. Asp190 and Asp191 in the poly-Asp repeat of LH3 that was suggested to be involved in catalysis are conserved (FIG. 3E, black arrows). A unique Trp145 in LH3 that was thought to be a gating residue is also conserved (FIG. 3E). Sequence alignment suggested that the R699 inter-domain loop has a 5-residue deletion (FIG. 3E) and lacks the cysteine-linked hairpin structure (FIG. 3E). The largest 14-residue deletion (FIG. 3E) occurs in the accessory domain that is not required for LH3's GGT activity but modulates LH2's GGT activity. These findings suggest the key residues involved in collagen GGT activity are conserved in R699.

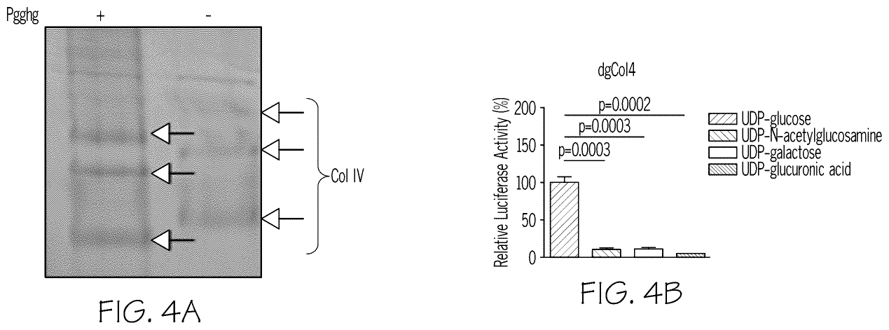

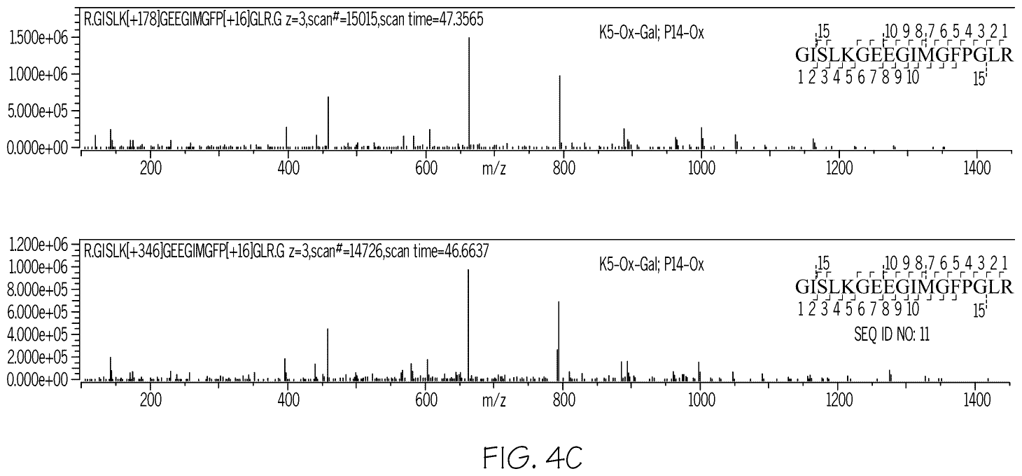

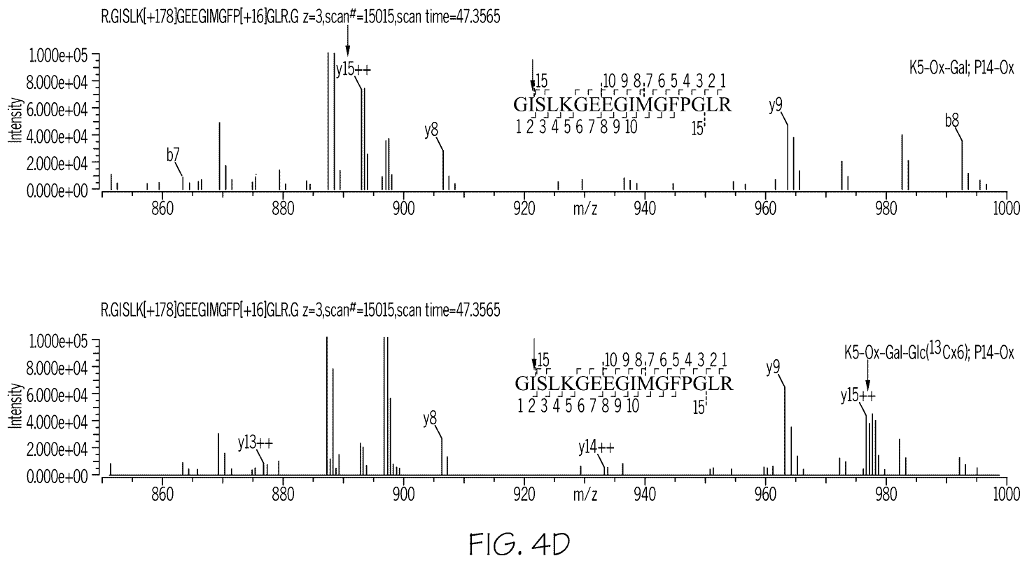

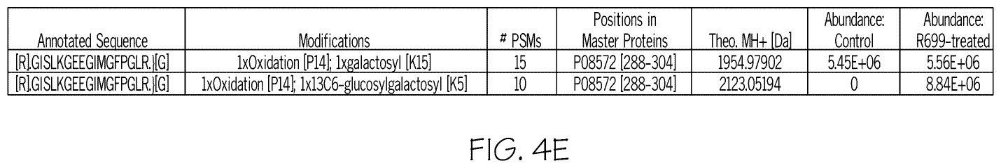

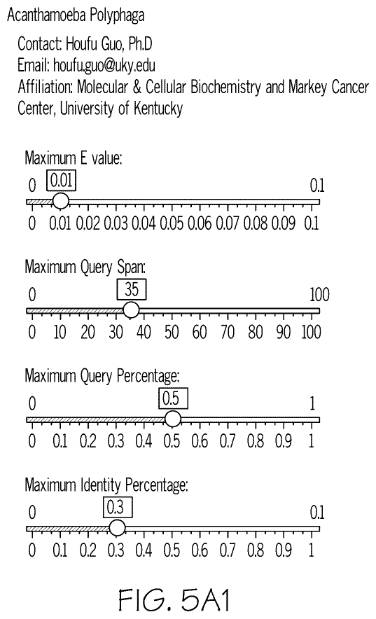

To test whether R699 is a collagen peptidyl GGT, R699 was reacted with deglucosylated type IV collagen substrate (FIG. 4A,4B) using UDP-glucose and 3 other sugar donors. Deglucosylation of type IV collagen was generated as we previously described (FIG. 4A). Under these conditions, R699 showed robust activity in the presence of UDP-glucose but no other sugar donors (FIG. 4B). The GGT enzymatic activity assay was also performed using UDP-[UL-¹³C₆]-glucose as a sugar donor to confirm the glucosylation events on type IV collagen by LC-MS (FIG. 4C-E). LC-MS analyses suggested that R699 (FIG. 4C,4D) glucosylates collagen peptidyl galactosylhydroxylysine to [¹³C₆]-glucosylgalactosylhydroxylysine. The MS/MS spectra exhibited peaks corresponding to b and y ion series from fragmentation of peptides (aa288-304 of Col4a2) containing galactosylhydroxylysine (top spectrum) or [¹³C₆]-glucosylgalactosylhydroxylysine (bottom spectrum). The b and y ion series were unambiguously identified and assigned. Enlarged spectra of the peptides in FIG. 4C showed the y15++ shift upon glucosylation (FIG. 4D, y15++ highlighted with arrows). The relative abundance quantification results suggested ¹³C₆]-glucosylgalactosylhydroxylysine is only present in the R699 treated deglucosylated type IV collagen sample but undetectable in untreated control (FIG. 4E), which is consistent with the luciferase-based enzymatic activity assay results (FIG. 4B). These findings suggested R699 is a mimiviral collagen GGT.

It was reported that mimiviral L230 functions as a collagen LH and a hydroxylysyl glucosyltransferase to produce peptidyl glucosylhydroxylysine27, thus there is the possibility of R699 modifying peptidyl glucosylhydroxylysine. Toward this end, recombinant L71 containing Hyl was produced by co-expressing L71 and L230 in Escherichia coli. L71 was then isolated and glucosylated by purified recombinant L230 using UDP-glucose as the sugar donor. L71 containing glucosylhydroxylysine was extensively dialyzed before reacting with R699. R699-catalyzed glucosylation reaction was detected with a luciferase-based assay. However, no luciferase activity was found (data not shown). These findings do not support that R699 functions as a peptidyl glucosylhydroxylysine glucosyltransferase. This work suggests that R699 acts on peptidyl galactosylhydroxylysine. The source of peptidyl galactosylhydroxylysine remains to be determined. It may be generated by the host or by an unknown mimiviral collagen hydroxylysyl galactosyltransferase. Since R655 shares moderate amino acid sequence identity (23%) with a human collagen hydroxylysyl galactosyltransferase GLT25D1, it warrants analysis of R655's collagen hydroxylysyl galactosyltransferase activity.

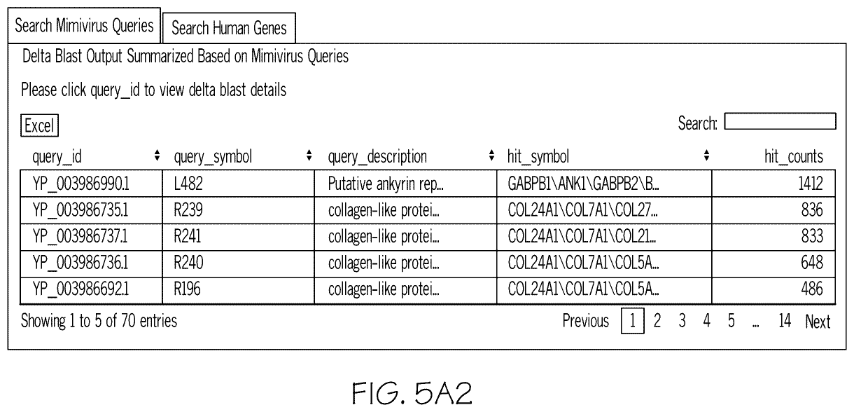

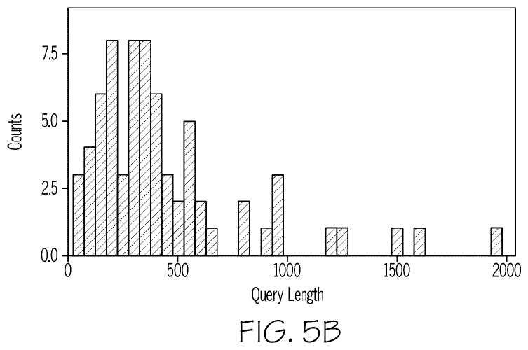

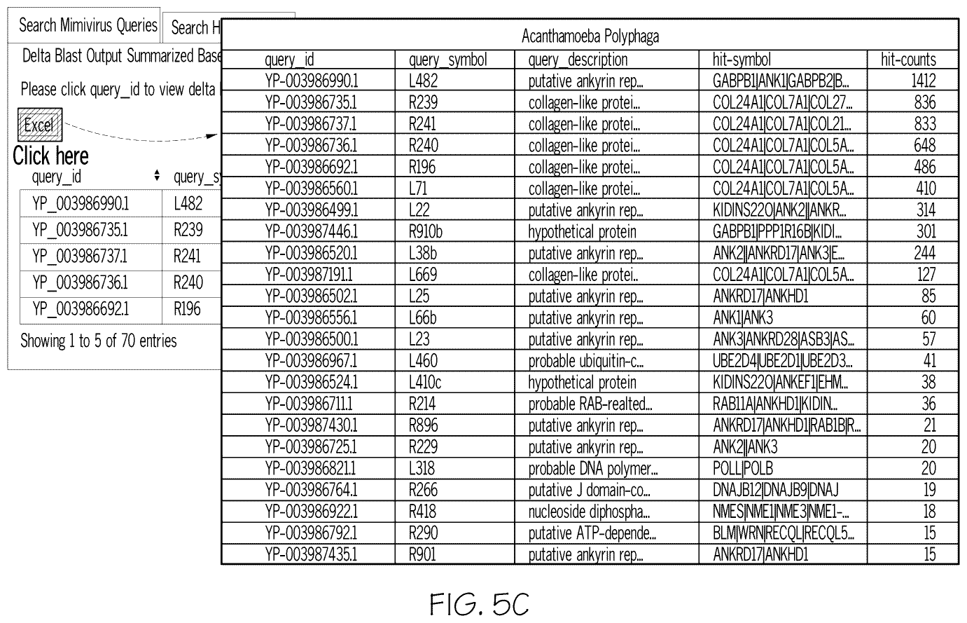

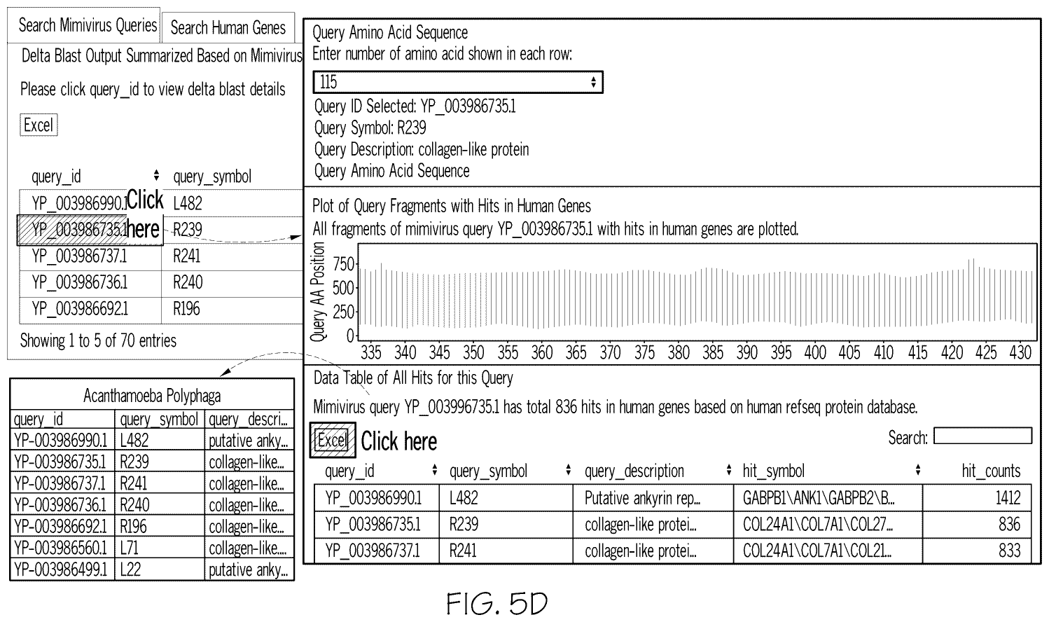

To facilitate the further analysis of the homology between humans and mimivirus, an interactive tool was established for easily searching and browsing of human and mimiviral homolog proteins (RRID Resource ID: SCR_022140 or guolab.shinyapps.io/app-mimivirus-publication/). Users can modify the search by changing the E value (Maximum Evalue), the length of query span in amino acid (Minimum Query Span) or percentage (Minimum QuerySpan Percent), the sequence identity percentage (Minimum Identity percentage) (FIG. 5A). The overall distribution of homologous proteins is shown in a histogram as counts vs. query length (FIG. 5B). If a list of all homologous proteins is needed, modify the search criteria without inputting a query. Clicking Excel on “Search Mimivirus Queries” tab or “Search Human Queries” tab will download an excel file including a list of all mimivirus or human homologous proteins, respectively (FIG. 5C). Search can be performed by inputting a query ID, gene name, or keywords about the query description. By clicking on the query_id, the details of the search (the Gene ID, symbol, description, and sequence) will be shown (FIG. 5D). The list of homologous hits is shown as a table under the Data table. The search details can be downloaded as an Excel file by clicking Excel (FIG. 5D).

In sum, following a genome-wide DELTA-BLAST of human and mimivirus genomes, 52 new mimiviral ORFs were found that may encode proteins with similarity to these of humans. To identify the potential functions of mimiviral ORFs, Gene Ontology (GO) and REACTOME pathway analyses were performed. The analyses showed that collagen and collagen-modifying enzymes form the largest subnetwork with most nodes. Two new putative glycosyltransferases, R655 and R699, were found in the mimiviral collagen-related pathways. Protein biochemical analyses confirmed that R699 is a new mimiviral collagen galactosylhydroxylysyl glucosyltransferase, suggesting the search is robust. An interactive and searchable genome-wide comparison tool (RRID Resource ID: SCR_022140 or guolab.shinyapps.io/app-mimivirus-publication/) has also been established. This tool is established based on the DELTA-BLAST results that helped us identify more homologous proteins. The interactive and searchable nature of the website allows the users to modify the search criteria and quickly browse human and mimivirus homologous proteins with different levels of homology at the genome-wide level.

### EXAMPLES

### Experimental Procedures

Comparative genome-wide analysis of human and mimivirus homologous proteins

To search mimiviral protein sequences against the human non-redundant protein sequence database, we installed and ran the DELTA-BLAST command line application in Lipscomb Compute Cluster at the University of Kentucky with default parameters. We obtained Acanthamoeba Polyphaga Mimivirus GCF_000888735.1 assembly and annotation data from NCBI's RefSeq ftp site ftp.ncbi.nlm.nih.gov/genomes/all/GCF/000/888/735/GCF_000888735.1_ViralProj60053. The file “GCF 000888735.1_ViralProj60053protein.faa.gz” that contains 979 protein sequences was used as the input. All mimiviral protein sequences were searched against the human non-redundant protein sequence database, which was downloaded from NCBI's blast ftp site ftp.ncbi.nlm.nih.gov/blast/db/. The file “GCF 000888735.1_ViralProj60053_feature_table.txt.gz” containing the feature information for all mimiviral protein sequences was used for annotation.

The DELTA-BLAST output in xml format was parsed and all high-scoring pairs (written as hits) were constructed into a tubular format using the biopython package Bio. Search10. The resulted data table was further processed in R. Among 979 mimiviral protein queries, 808 queries had at least one hit and 556603 hits were found in total. Using e value<=0.01, hit span>=35 amino acids, and the percentage of identical sequences between query and hit>=0.25 as cutoffs, 356 query sequences with 85881 total hits passed the defined criteria. These 356 query sequences share sequence similarity with 4123 unique human RefSeq records in total.

To confirm the similarity mapping between mimivirus and human protein sequences, 4123 unique human RefSeq protein records we identified were DELTA-BLAST searched against the mimivirus non-redundant protein sequence database. The human RefSeq protein sequence file “GCF_000001405.39_GRCh38.p13_protein.faa.gz” of the latest GRCh38 assembly was downloaded from NCBI's RefSeq ftp site ftp.ncbi.nlm.nih.gov/genomes/refseq/vertebrate_mammalian/Homo_sapiens/latest_assembly_versions/GCF_000001405.39_GRCh38.p13. Four RefSeq records were not present in the file therefore manually checked for their sequences in NCBI. A newly formed file containing all 4123 protein sequences of interest was used for DELTA-BLAST command line application with default parameters. After DELTA-BLAST, the output was processed in the same manner as the first DELTA-BLAST search. Using the same cutoffs, we found that 3325 human RefSeq records (1049 unique gene symbols) have hits in 307 unique mimiviral protein sequences. Of these 307 unique mimiviral protein sequences, 265 of them overlap with the 322 mimiviral queries that we identified in the first round of search. To summarize the results for overlapping mimiviral sequences, 1031 human genes and their corresponding mimiviral sequences were organized and presented.

### Enrichmentmap and Network

To reduce the redundant hits between different databases, we selected the hits from the RefSeq database to perform pathway enrichment analysis using HUGO gene symbol, which resulted in 322 queries with at least one hit and 41520 hits in total. At the protein level, these 41520 hits are from 4123 unique RefSeq records, which contains 2027 proteins (IDs prefix with NP) and 2096 predicted proteins (IDs prefix with XP). These RefSeq record IDs were then converted into HUGO gene symbols using Bioconductor package biomaRt. The ones that cannot be converted by biomaRt were manually checked for the corresponding gene symbols in Genecard and BioGPS. Eventually, this conversion resulted in 1236 unique gene symbols, which were then used for pathway enrichment analysis and building Shiny App for visualization. The Shiny App is hosted on shinyapps.io server and is publicly available (guolab.shinyapps.io/app-mimivirus-publication/). This resource was submitted to RRID Portal with a Resource ID: SCR_022140.

To understand the overall biological and biochemical processes that the hits may be involved in, pathway enrichment analysis was performed using the R package gprofiler2. The significant GO and REACTOME pathways (adjusted p-value<=0.05) with term sizes between 5 and 350 were selected for constructing pathway networks using EnrichmentMap (45). The resulted clusters were then automatically defined and summarized into major biological themes using AutoAnnotate. Finally, collagen-related pathways which formed the largest subnetwork were presented separately. All three steps were performed in Cytoscape 3.8.2.

Cloning, expression, and purification of R699 and variants

R699 gene was synthesized (Genscript). For enzymatic activity assay, R699 was cloned into a modified version of the pET28 vector using BamH1 and EcoR1 sites. This modified version of pET28 has PreScission and BamH1 recognition sites inserted to replace the thrombin recognition site. The endogenous BamH1 site was destroyed. Mutant constructs were generated using QuickChange Lightning Site-Directed Mutagenesis Kit (Agilent). For crystallization, R699 was cloned into a version of pET28-mCherry vector using BamH1 and EcoR1 sites. This pET28-mCherry vector has mCherry gene sequence and PreScission recognition site inserted between Nhe1 and BamH1 sites. All plasmids were verified by sanger sequencing and transformed into E. coli BL21 (NEB) for protein expression. Small scale R699-BL21 overnight culture with 50 mg per liter of kanamycin (GoldBio) was prepared and 10 ml of small-scale overnight culture was used to inoculate 800 ml large scale culture using Terrific Broth Medium (Alpha Biosciences) in the presence of the same amount of kanamycin. Culture was grown at 37° C. to OD₆₀₀=1.5, induced with 1 mM isopropyl β-D-1-thiogalactopyranoside (IPTG, GoldBio) and grown at 16° C. for 18 hours. Cells were collected, pelleted and then resuspended in binding buffer (20 mM Tris, pH 8.0, 200 mM NaCl and 15 mM imidazole). The cells were lysed by sonication and then centrifuged at 23,000 g for 15 min. The recombinant R699 proteins (wild type or mutants) were purified with immobilized metal affinity chromatography and eluted with elution buffer (200 mM NaCl and 300 mM imidazole, pH 8.0). For enzymatic activity assay, R699 protein was dialyzed at 16° C. for 18 hours in 20 mM HEPES, pH 7.4, 150 mM NaCl.

Crystallization, structure determination, and refinement

mCherry-R699 was first purified with immobilized metal affinity chromatography as described above. The eluted recombinant protein was cleaved with PreScission protease at 4° C. for 18 hours while dialyzing in gel filtration buffer (20 mM Tris, pH 8.0, 200 mM NaCl). After PreScission protease cleavage, R699 was purified with reverse immobilized metal affinity chromatography to remove mCherry protein and other contaminants that bind to nickel resin. The eluted protein was further separated by gel filtration using a Hiload 16/60 Superdex 200 PG column at a flow rate of 1 ml per minute. Peak fractions were combined and concentrated for crystal trials. Single, high-quality crystals with two molecules in the asymmetric unit were obtained via hanging drop vapor diffusion using a Mosquito liquid handling robot (TTPLabtech) using a 200-nL drop. R699 (16 mg/mL) supplemented with 10 mM uracil-diphosphate glucose and 2 mM manganese(II) chloride was mixed with 200 mM NaCl, 0.1M Na/K phosphate pH 6.2 and 40% (v/v) PEG 400 at 1:1 ratio and incubated at 18° C. Diffraction data were collected on the 22-ID beamline of SERCAT at the Advanced Photon Source, Argonne National Laboratory at 110K at a wavelength of 1.0 Å. Data were processed using CCP4, version 7.1.018 and the structure was solved by molecular replacement with Phenix using RoseTTAFold and AlphaFold models as search templates. The structure was then fully built and refined via iterative model building and refinement using Coot and Phenix, respectively. Protein structure similarity was compared using the Dali server. Structure interface was analyzed using protein interfaces, surfaces and assemblies' service PISA at the European Bioinformatics Institute. Molecular graphics were prepared using Pymol (DeLano, W. L The PyMOL molecular graphics system. www.pymol.org). Amino acid sequence alignment of R699 and human GGTs was performed using Clustal Omega.

GGT enzymatic activity assay

GGT activity was measured similarly as previously described (12). The assay was performed in reaction buffer (100 mM HEPES buffer pH 8.0, 150 mM NaCl) at 37° C. for 1 h with 1 μM R699 enzyme, 100 μM MnCl₂, 200 μM UDP-glucose (MilliporeSigma, St. Louis, MO), 1 mM dithiothreitol and 1.75 mM galactosyl hydroxylysine (Gal-Hyl, Cayman Chemical, Ann Arbor, MI) or 2 μM deglucosylated collagen IV. Deglucosylated collagen IV was generated using a glycosidase PGGHG as previously described (12). GGT activity was measured by detecting UDP production with an ATP-based luciferase assay (UDP-Glo™ Glycosyltransferase Assay, Promega, Madison, WI) according to manufacturers' instructions. Experiments were performed in triplicate from distinct samples, and an unpaired t-test was used to compare the enzymatic activity of different samples. The glucosylation of galactosyl hydroxylysine was further confirmed by mass spectrometry.

### Mass Spectrometry

To confirm the glucosylation of Gal-Hyl by R699, the R699 GGT assay was performed similarly as discussed above, except that UDP-glucose was replaced with the same concentration of UDP-[UL-¹³C₆] glucose (Omicron Biochemicals, Inc). LC-MS analysis was used to detect [13C]GlcGal-Hyl. LH3 catalyzed GGT activity assay was used as a positive control. For LC-MS analysis, R699 and LH3 assay samples were diluted to ˜1 μM in 50% acetonitrile containing 0.1% formic acid. LC-MS analysis was performed using a 1260 Infinity UHPLC System (Agilent) coupled to a Qtrap 6500 mass spectrometer (SCIEX). Samples were separated on a Kinetex EVO C18 column (Phenomenex) with mobile phases included: A) water+0.1% formic acid, B) acetonitrile+0.1% formic acid. LC peaks were integrated using MultiQuant software (SCIEX). Peak areas and chromatograms were plotted using custom R scripts. Experiments were performed once.

### Circular Dichroism

Circular dichroism spectra were measured using a J-810 spectrapolarimeter (Jasco, Easton, MD) with a 2 mm path length quartz cuvette. All measurements were performed at 20° C. Three scans were averaged to generate each spectrum. A blank spectrum of buffer was collected in the same manner and used for background subtraction. For FIG. 3E, R699 wild type and mutant recombinant proteins were dialyzed and measured in 20 mM HEPES and 150 mM NaCl (pH 7.4). For FIG. 4D, R699 recombinant proteins were analyzed in 0.01 M sodium phosphate, 150 mM NaCl (pH 7.4) and 10% glycerol at a concentration of 0.5 mg ml⁻¹ Results represent the mean values from triplicate technical repeats in a single experiment. Each protein was analyzed once.

Data availability Crystal structure has been deposited in the Worldwide Protein Data Bank under RCSB accession ID number 7UL9.

Various modifications of the present disclosure, in addition to those shown and described herein, will be apparent to those skilled in the art of the above description. Such modifications are also intended to fall within the scope of the appended claims.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The drawings are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

### REFERENCES

1. Frantz, C., Stewart, K. M., and Weaver, V. M. (2010) The extracellular matrix at a glance. J Cell Sci 123, 4195-4200
2. Hynes, R. O. (2009) The extracellular matrix: not just pretty fibrils. Science 326, 1216-1219
3. Ricard-Blum, S. (2011) The collagen family. Cold Spring Harb Perspect Biol 3, a004978
4. Gjaltema, R. A., and Bank, R. A. (2017) Molecular insights into prolyl and lysyl hydroxylation of fibrillar collagens in health and disease. Crit Rev Biochem Mol Biol 52, 74-95
5. Yamauchi, M., and Sricholpech, M. (2012) Lysine post-translational modifications of collagen. Essays Biochem 52, 113-133
6. Pornprasertsuk, S., Duarte, W. R., Mochida, Y., and Yamauchi, M. (2004) Lysyl hydroxylase-2b directs collagen cross-linking pathways in MC3T3-E1 cells. J Bone Miner Res 19, 1349-1355
7. Terajima, M., Taga, Y., Sricholpech, M., Kayashima, Y., Sumida, N., Maeda, N., Hattori, S., and Yamauchi, M. (2019) Role of Glycosyltransferase 25 Domain 1 in Type I Collagen Glycosylation and Molecular Phenotypes. Biochemistry 58, 5040-5051
8. Sricholpech, M., Perdivara, I., Nagaoka, H., Yokoyama, M., Tomer, K. B., and Yamauchi, M. (2011) Lysyl hydroxylase 3 glucosylates galactosylhydroxylysine residues in type I collagen in osteoblast culture. J Biol Chem 286, 8846-8856
9. Sricholpech, M., Perdivara, I., Yokoyama, M., Nagaoka, H., Terajima, M., Tomer, K. B., and Yamauchi, M. (2012) Lysyl hydroxylase 3-mediated glucosylation in type I collagen: molecular loci and biological significance. J Biol Chem 287, 22998-23009
10. Hennet, T. (2019) Collagen glycosylation. Curr Opin Struct Biol 56, 131-138
11. Schegg, B., Hulsmeier, A. J., Rutschmann, C., Maag, C., and Hennet, T. (2009) Core glycosylation of collagen is initiated by two beta (1-O) galactosyltransferases. Mol Cell Biol 29, 943-952
12. Guo, H. F., Bota-Rabassedas, N., Terajima, M., Leticia Rodriguez, B., Gibbons, D. L., Chen, Y., Banerjee, P., Tsai, C. L., Tan, X., Liu, X., Yu, J., Tokmina-Roszyk, M., Stawikowska, R., Fields, G. B., Miller, M. D., Wang, X., Lee, J., Dalby, K. N., Creighton, C. J., Phillips, G. N., Jr., Tainer, J. A., Yamauchi, M., and Kurie, J. M. (2021) A collagen glucosyltransferase drives lung adenocarcinoma progression in mice. Commun Biol 4, 482
13. Yamauchi, M., Barker, T. H., Gibbons, D. L., and Kurie, J. M. (2018) The fibrotic tumor stroma. J Clin Invest 128, 16-25
14. Scietti, L., Campioni, M., and Forneris, F. (2019) SiMPLOD, a Structure-Integrated Database of Collagen Lysyl Hydroxylase (LH/PLOD) Enzyme Variants. J Bone Miner Res 34, 1376-1382
15. Hyland, J., Ala-Kokko, L., Royce, P., Steinmann, B., Kivirikko, K. I., and Myllyla, R. (1992) A homozygous stop codon in the lysyl hydroxylase gene in two siblings with Ehlers-Danlos syndrome type VI. Nat Genet 2, 228-231
16. Ha-Vinh, R., Alanay, Y., Bank, R. A., Campos-Xavier, A. B., Zankl, A., Superti-Furga, A., and Bonafe, L. (2004) Phenotypic and molecular characterization of Bruck syndrome (osteogenesis imperfecta with contractures of the large joints) caused by a recessive mutation in PLOD2. Am J Med Genet A 131, 115-120
17. Vahidnezhad, H., Youssefian, L., Saeidian, A. H., Touati, A., Pajouhanfar, S., Baghdadi, T., Shadmehri, A. A., Giunta, C., Kraenzlin, M., Syx, D., Malfait, F., Has, C., Lwin, S. M., Karamzadeh, R., Liu, L., Guy, A., Hamid, M., Kariminejad, A., Zeinali, S., McGrath, J. A., and Uitto, J. (2019) Mutations in PLOD3, encoding lysyl hydroxylase 3, cause a complex connective tissue disorder including recessive dystrophic epidermolysis bullosa-like blistering phenotype with abnormal anchoring fibrils and type VII collagen deficiency. Matrix Biol 81, 91-106
18. Salo, A. M., Cox, H., Farndon, P., Moss, C., Grindulis, H., Risteli, M., Robins, S. P., and Myllyla, R. (2008) A connective tissue disorder caused by mutations of the lysyl hydroxylase 3 gene. Am J Hum Genet 83, 495-503
19. Chen, Y., Terajima, M., Yang, Y., Sun, L., Ahn, Y. H., Pankova, D., Puperi, D. S., Watanabe, T., Kim, M. P., Blackmon, S. H., Rodriguez, J., Liu, H., Behrens, C., Wistuba, II, Minelli, R., Scott, K. L., Sanchez-Adams, J., Guilak, F., Pati, D., Thilaganathan, N., Burns, A. R., Creighton, C. J., Martinez, E. D., Zal, T., Grande-Allen, K. J., Yamauchi, M., and Kurie, J. M. (2015) Lysyl hydroxylase 2 induces a collagen cross-link switch in tumor stroma. J Clin Invest 125, 1147-1162
20. Levental, K. R., Yu, H., Kass, L., Lakins, J. N., Egeblad, M., Erler, J. T., Fong, S. F., Csiszar, K., Giaccia, A., Weninger, W., Yamauchi, M., Gasser, D. L., and Weaver, V. M. (2009) Matrix crosslinking forces tumor progression by enhancing integrin signaling. Cell 139, 891-906
21. Eisinger-Mathason, T. S., Zhang, M., Qiu, Q., Skuli, N., Nakazawa, M. S., Karakasheva, T., Mucaj, V., Shay, J. E., Stangenberg, L., Sadri, N., Pure, E., Yoon, S. S., Kirsch, D. G., and Simon, M. C. (2013) Hypoxia-dependent modification of collagen networks promotes sarcoma metastasis. Cancer Discov 3, 1190-1205
22. Cabral, W. A., Chang, W., Barnes, A. M., Weis, M., Scott, M. A., Leikin, S., Makareeva, E., Kuznetsova, N. V., Rosenbaum, K. N., Tifft, C. J., Bulas, D. I., Kozma, C., Smith, P. A., Eyre, D. R., and Marini, J. C. (2007) Prolyl 3-hydroxylase 1 deficiency causes a recessive metabolic bone disorder resembling lethal/severe osteogenesis imperfecta. Nat Genet 39, 359-365
23. Lukomski, S., Bachert, B. A., Squeglia, F., and Berisio, R. (2017) Collagen-like proteins of pathogenic streptococci. Mol Microbiol 103, 919-930
24. Price, S., and Anandan, S. (2013) Characterization of a novel collagen-like protein TrpA in the cyanobacterium Trichodesmium erythraeum IMS101. J Phycol 49, 758-764
25. Luther, K. B., Hulsmeier, A. J., Schegg, B., Deuber, S. A., Raoult, D., and Hennet, T. (2011) Mimivirus collagen is modified by bifunctional lysyl hydroxylase and glycosyltransferase enzyme. J Biol Chem 286, 43701-43709
26. Celerin, M., Ray, J. M., Schisler, N. J., Day, A. W., Stetler-Stevenson, W. G., and Laudenbach, D. E. (1996) Fungal fimbriae are composed of collagen. EMBO J 15, 4445-4453
27. Raoult, D., Audic, S., Robert, C., Abergel, C., Renesto, P., Ogata, H., La Scola, B., Suzan, M., and Claverie, J. M. (2004) The 1.2-megabase genome sequence of Mimivirus. Science 306, 1344-1350
28. Rutschmann, C., Baumann, S., Cabalzar, J., Luther, K. B., and Hennet, T. (2014) Recombinant expression of hydroxylated human collagen in Escherichia coli. Appl Microbiol Biotechnol 98, 4445-4455
29. Guo, H. F., Tsai, C. L., Terajima, M., Tan, X., Banerjee, P., Miller, M. D., Liu, X., Yu, J., Byemerwa, J., Alvarado, S., Kaoud, T. S., Dalby, K. N., Bota-Rabassedas, N., Chen, Y., Yamauchi, M., Tainer, J. A., Phillips, G. N., Jr., and Kurie, J. M. (2018) Pro-metastatic collagen lysyl hydroxylase dimer assemblies stabilized by Fe(2+)-binding. Nat Commun 9, 512
30. John, D. C., Watson, R., Kind, A. J., Scott, A. R., Kadler, K. E., and Bulleid, N. J. (1999) Expression of an engineered form of recombinant procollagen in mouse milk. Nat Biotechnol 17, 385-389
31. Vuorela, A., Myllyharju, J., Nissi, R., Pihlajaniemi, T., and Kivirikko, K. I. (1997) Assembly of human prolyl 4-hydroxylase and type III collagen in the yeast Pichia pastoris: formation of a stable enzyme tetramer requires coexpression with collagen and assembly of a stable collagen requires coexpression with prolyl 4-hydroxylase. EMBO J 16, 6702-6712
32. Shoseyov, O., Posen, Y., and Grynspan, F. (2013) Human recombinant type I collagen produced in plants. Tissue Eng PartA 19, 1527-1533
33. Gupta, A., Lad, S. B., Ghodke, P. P., Pradeepkumar, P. I., and Kondabagil, K. (2019) Mimivirus encodes a multifunctional primase with DNA/RNA polymerase, terminal transferase and translesion synthesis activities. Nucleic Acids Res 47, 6932-6945
34. Zinoviev, A., Kuroha, K., Pestova, T. V., and Hellen, C. U. T. (2019) Two classes of EF1-family translational GTPases encoded by giant viruses. Nucleic Acids Res 47, 5761-5776
35. Bekliz, M., Azza, S., Seligmann, H., Decloquement, P., Raoult, D., and La Scola, B. (2018) Experimental Analysis of Mimivirus Translation Initiation Factor 4a Reveals Its Importance in Viral Protein Translation during Infection of Acanthamoeba polyphaga. J Virol 92
36. Jeudy, S., Lartigue, A., Claverie, J. M., and Abergel, C. (2009) Dissecting the unique nucleotide specificity of mimivirus nucleoside diphosphate kinase. J Virol 83, 7142-7150
37. Benarroch, D., Qiu, Z. R., Schwer, B., and Shuman, S. (2009) Characterization of a mimivirus RNA cap guanine-N2 methyltransferase. RNA 15, 666-674
38. Legendre, M., Santini, S., Rico, A., Abergel, C., and Claverie, J. M. (2011) Breaking the 1000-gene barrier for Mimivirus using ultra-deep genome and transcriptome sequencing. Virol J 8, 99
39. Aherfi, S., Brahim Belhaouari, D., Pinault, L., Baudoin, J. P., Decloquement, P., Abrahao, J., Colson, P., Levasseur, A., Lamb, D. C., Chabriere, E., Raoult, D., and La Scola, B. (2022) Incomplete tricarboxylic acid cycle and proton gradient in Pandora virus massiliensis: is it still a virus? ISME J 16, 695-704
40. Blanc-Mathieu, R., Dahle, H., Hofgaard, A., Brandt, D., Ban, H., Kalinowski, J., Ogata, H., and Sandaa, R. A. (2021) A persistent giant algal virus, with a unique morphology, encodes an unprecedented number of genes involved in energy metabolism. J Virol
41. Boratyn, G. M., Schaffer, A. A., Agarwala, R., Altschul, S. F., Lipman, D. J., and Madden, T. L. (2012) Domain enhanced lookup time accelerated BLAST. Biol Direct 7, 12
42. Shah, N., Hulsmeier, A. J., Hochhold, N., Neidhart, M., Gay, S., and Hennet, T. (2014) Exposure to mimivirus collagen promotes arthritis. J Virol 88, 838-845
43. Scietti, L., Chiapparino, A., De Giorgi, F., Fumagalli, M., Khoriauli, L., Nergadze, S., Basu, S., Olieric, V., Cucca, L., Banushi, B., Profumo, A., Giulotto, E., Gissen, P., and Forneris, F. (2018) Molecular architecture of the multifunctional collagen lysyl hydroxylase and glycosyltransferase LH3. Nat Commun 9, 3163
44. Rautavuoma, K., Takaluoma, K., Passoja, K., Pirskanen, A., Kvist, A. P., Kivirikko, K. I., and Myllyharju, J. (2002) Characterization of three fragments that constitute the monomers of the human lysyl hydroxylase isoenzymes 1-3. The 30-kDa N-terminal fragment is not required for lysyl hydroxylase activity. J Biol Chem 277, 23084-23091
45. Merico, D., Isserlin, R., Stueker, O., Emili, A., and Bader, G. D. (2010) Enrichment map: a network-based method for gene-set enrichment visualization and interpretation. PLoS One 5, e13984
46. Kucera, M., Isserlin, R., Arkhangorodsky, A., and Bader, G. D. (2016) AutoAnnotate: A Cytoscape app for summarizing networks with semantic annotations. F1000Res 5, 1717
47. Shannon, P., Markiel, A., Ozier, O., Baliga, N. S., Wang, J. T., Ramage, D., Amin, N., Schwikowski, B., and Ideker, T. (2003) Cytoscape: a software environment for integrated models of biomolecular interaction networks. Genome Res 13, 2498-2504
48. Collaborative Computational Project, N. (1994) The CCP4 suite: programs for protein crystallography. Acta Crystallogr D Biol Crystallogr 50, 760-763
49. Baek, M., DiMaio, F., Anishchenko, I., Dauparas, J., Ovchinnikov, S., Lee, G. R., Wang, J., Cong, Q., Kinch, L. N., Schaeffer, R. D., Millan, C., Park, H., Adams, C., Glassman, C. R., DeGiovanni, A., Pereira, J. H., Rodrigues, A. V., van Dijk, A. A., Ebrecht, A. C., Opperman, D. J., Sagmeister, T., Buhlheller, C., Pavkov-Keller, T., Rathinaswamy, M. K., Dalwadi, U., Yip, C. K., Burke, J. E., Garcia, K. C., Grishin, N. V., Adams, P. D., Read, R. J., and Baker, D. (2021) Accurate prediction of protein structures and interactions using a three-track neural network. Science 373, 871-876
50. Jumper, J., Evans, R., Pritzel, A., Green, T., Figurnov, M., Ronneberger, O., Tunyasuvunakool, K., Bates, R., Zidek, A., Potapenko, A., Bridgland, A., Meyer, C., Kohl, S. A. A., Ballard, A. J., Cowie, A., Romera-Paredes, B., Nikolov, S., Jain, R., Adler, J., Back, T., Petersen, S., Reiman, D., Clancy, E., Zielinski, M., Steinegger, M., Pacholska, M., Berghammer, T., Bodenstein, S., Silver, D., Vinyals, O., Senior, A. W., Kavukcuoglu, K., Kohli, P., and Hassabis, D. (2021) Highly accurate protein structure prediction with AlphaFold. Nature 596, 583-589
51. Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., McCoy, A. J., Moriarty, N. W., Oeffner, R., Read, R. J., Richardson, D. C., Richardson, J. S., Terwilliger, T. C., and Zwart, P. H. (2010) PHENIX: a comprehensive Python-based system for macromolecular structure solution. Acta Crystallogr D Biol Crystallogr 66, 213-221
52. Emsley, P., and Cowtan, K. (2004) Coot: model-building tools for molecular graphics. Acta Crystallogr D Biol Crystallogr 60, 2126-2132
53. Holm, L. (2020) Using Dali for Protein Structure Comparison. Methods Mol Biol 2112, 29-42
54. Krissinel, E., and Henrick, K. (2007) Inference of macromolecular assemblies from crystalline state. J Mol Biol 372, 774-797
55. Madeira, F., Park, Y. M., Lee, J., Buso, N., Gur, T., Madhusoodanan, N., Basutkar, P., Tivey, A. R. N., Potter, S. C., Finn, R. D., and Lopez, R. (2019) The EMBL-EBI search and sequence analysis tools APIs in 2019. Nucleic acids research 47, W636-W641

We claim:

1. A system for producing collagen comprising a bacterial cell expressing an enzyme with at least 85% identity to an amino acid sequence as set forth in at least one of SEQ ID NOs: 1, 7, 8, 9, 12, or 13.

2. The system of claim 1, wherein the bacterial cell expresses SEQ ID NO: 1.

3. The system of claim 1, wherein the bacterial cell expresses SEQ ID NO: 13.

4. The system of claim 1, wherein the bacterial cell expresses SEQ ID NO: 7.

5. The system of claim 1, wherein the bacterial cell expresses SEQ ID NO: 8.

6. The system of claim 1, wherein the bacterial cell expresses SEQ ID NO: 9.

7. The system of claim 1, wherein the bacterial cell is an Escherichia coli cell.

8. The system of claim 1, wherein the bacterial cell further expresses a human collagen protein or fragment thereof.

9. The system of claim 1, further comprising a human collagen protein.

10. A method for modifying a collagen protein comprising contacting a collagen protein with a recombinant galactosylhydroxylysyl glucosyltransferase (GGT) derived from Acanthamoeba polyphaga mimivirus.

11. The method of claim 10, wherein the GGT is is selected from the group consisting of SEQ ID NOs: 1, 7, 8, 9, 12, and 13.

12. The method of claim 11, wherein the GGT comprises SEQ ID NO: 13.

13. The method of claim 10, wherein the GGT is recombinantly expressed in a bacterial cell.

14. The method of claim 13, wherein the bacterial cell is an Escherichia coli cell.

15. The method of claim 13, wherein the GGT is expressed from a nuclei acid encoding the GGT within the bacterial cell.
